(19) United States
(12) Patent Application Publication
(10) Pub. No.: US 2019/0276751 A1
(43) Pub. Date: Sep. 12, 2019

(54) NORMAL PARAFFIN COMPOSITION

(71) Applicant: SHELL OIL COMPANY, HOUSTON, TX (US)

(72) Inventor: Gerrit Leendert BEZEMER, Amsterdam (NL)

(21) Appl. No.: 16/346,780

(22) PCT Filed: Nov. 2, 2017
(86) PCT No.: PCT/EP2017/077996

§ 371 Date: May 1, 2019

(30) Foreign Application Priority Data
Nov. 7, 2016 (EP) 16197552.9

(51) Int. Cl. C10G 45/02; C07C 303/26; C08L 91/06; C10G 49/22

(52) U.S. Cl. CPC C10G 45/02; C07C 303/26; C08L 91/06; C10G 2300/308; C10G 2300/1022; C10G 2300/302; C10G 2300/304; C10G 49/22

(57) ABSTRACT

The present invention relates to a normal paraffin composition comprising from 45 to 60 wt. % of a fraction of normal paraffin having from 10 to 13 carbon atoms and from 40 to 55 wt. % of a fraction of normal paraffin having from 14 to 18 carbon atoms.

### FIELD OF THE INVENTION

The present invention relates to a normal paraffin composition and a process to prepare the normal paraffin composition.

### BACKGROUND TO THE INVENTION

Normal paraffins may be obtained by various processes. EP2655565 disclose a method for deriving paraffins from crude oil. Also, paraffins may be obtained using the so called Fischer-Tropsch process. An example of such process is disclosed in WO2014095814 and WO2016107864.

WO2016107864 discloses a process to prepare paraffins and waxes. In WO2016107864 a Fischer-Tropsch product stream comprising paraffins having from 10 to 300 carbon atoms is subjected to a hydrogenation step followed by separation of the hydrogenated Fischer-Tropsch product to obtain at least a fraction comprising 10 to 17 carbon atoms.

A problem of the process disclosed in WO2016107864 is that the hydrocarbon chain length distribution of the normal paraffin composition fraction comprising 10 to 17 carbon is such that the ratio between fraction having from 10 to 13 carbon atoms and the fraction of normal paraffin having from 14 to 17 carbon atoms in the normal paraffin composition is about 1. The fraction of normal paraffins having from 14 to 17 carbon atoms, however, is a less favorable product than the fraction of normal paraffin having from 10 to 13 carbon atoms. The demand for linear alkyl benzene sulphonate (LAS) comprising the normal paraffin fraction having from 10 to 13 carbon atoms is much higher than the demand for paraffin sulphonates and chloroparaffins, both comprising the normal paraffin fraction having from 14 to 17 carbon atoms.

It is an object of the invention to solve or minimize at least one of the above problems. It is a further object of the present invention to provide a normal paraffin composition which can be advantageously used in the manufacture of linear alkyl benzene (LAB), an intermediate for the production of LAS, the most widely used surfactants in the detergent industry.

Moreover, an object of the present invention is to provide an a more efficient and simple method to prepare normal paraffins having a higher amount of the fraction comprising 10 to 13 carbon atoms on a smaller scale.

One of the above or other objects may be achieved according to the present invention by providing a normal paraffin composition comprising from 45 to 60 wt. % of a fraction of normal paraffin having from 10 to 13 carbon atoms and from 40 to 55 wt. % of a fraction of normal paraffin having from 14 to 18 carbon atoms.

It has been found according to the present invention that the normal paraffin composition may be advantageously used for the production of surfactants in the detergent industry.

In another embodiment of the present invention there is provided a process to prepare normal paraffin compositions.

An advantage of the process according to the present invention is that the process line-up has been simplified and that there is less risk of corrosion of the distillation columns, heat exchanger and additional equipment.

A further advantage is that by selecting the light wax stream the equipment size gets smaller and the separation easier. In this way the production of normal paraffins is also attractive on smaller scale. Yet a further advantage is that less energy is required for the heating and distillation.

Another advantage is that the composition of the normal paraffins can be influenced such that the normal paraffins composition will be lighter because the heavier molecules reside in the heavy wax.

### DETAILED DESCRIPTION OF THE INVENTION

The following detailed description does not limit the invention. Instead, the scope of the invention is defined by the appended claims.

According to the present invention, a normal paraffin composition comprises from 45 to 60 wt. % of a fraction of normal paraffin having from 10 to 13 carbon atoms and from 40 to 55 wt. % of a fraction of normal paraffin having from 14 to 18 carbon atoms.

Normal paraffin compositions are known and described for example in WO2014095814 and WO2016107864.

Suitably, the normal paraffin composition according to the present invention comprises 49 wt. % of the fraction of normal paraffin having from 10 to 13 carbon atoms and 51 wt. % of a fraction of normal paraffin having from 14 to 18 carbon atoms based on the total amount of the normal paraffin composition.

Preferably, the normal paraffin composition according to the present invention comprises a fraction of normal paraffin having from 10 to 13 carbon atoms comprises 10 carbon atoms in the range of from 10 to 11 wt. %, 11 carbon atoms in the range of from 30 to 32 wt. %, 12 carbon atoms in a range of from 30 to 32 wt. % and 13 carbon atoms in a range of from 23 to 26 wt. % based on the amount of the normal paraffin having from 10 to 13 carbon atoms.

Also, the normal paraffin composition according to the present invention comprises the fraction of normal paraffin having from 14 to 18 carbon atoms comprises 14 carbon atoms in a range of from 25 to 27 wt. %, 15 carbon atoms in a range of from 24 to 26 wt. %, 16 carbon atoms in a range of from 22 to 23 wt. %, 17 carbon atoms in a range of from 18 to 20 wt. % and 18 carbon atoms in a range of from 4 to 6 wt. % based on the amount of the normal paraffin having from 14 to 18 carbon atoms.

It is preferred that the normal paraffin composition according to the present invention is a Fischer-Tropsch derived normal paraffin composition.

The Fischer-Tropsch derived normal paraffin composition is derived from a Fischer-Tropsch process. Fischer-Tropsch product stream is known in the art. By the term “Fischer-Tropsch derived” is meant a paraffin wax is, or is derived from a Fischer-Tropsch process. A Fischer-Tropsch derived normal paraffin composition may also be referred to a GTL (Gas-to-Liquids) product. An example of a Fischer-Tropsch process is given in WO2002/102941, EP 1 498 469 and WO2004/009739, the teaching of which is incorporated by reference.

The Fischer-Tropsch derived normal paraffin composition comprises paraffins, primarily n-paraffins. Preferably, the Fischer-Tropsch derived normal paraffin composition comprises more than 85 wt. % of n-paraffins, preferably more than 90 wt. % of n-paraffins.

In a further aspect, the present invention provides a process to prepare a Fischer-Tropsch derived normal paraffin composition, the process at least comprising the following steps:
(a) providing a Fischer-Tropsch product stream;
(b) separating the Fischer-Tropsch product stream of step (a), thereby obtaining a gaseous hydrocarbon stream and a first liquid hydrocarbon stream;
(c) cooling of the gaseous hydrocarbon stream of step (b) in one or more steps to obtain a second liquid hydrocarbon stream and a third liquid hydrocarbon stream;
(d) subjecting the second and third liquid hydrocarbon streams of step (c) to a hydrogenation step, thereby obtaining a hydrogenated liquid hydrocarbon stream;
(e) separating the hydrogenated liquid hydrocarbon stream of step (d) by one or more atmospheric distillation(s), thereby obtaining at least a normal paraffin composition comprising a fraction of normal paraffin fraction comprising 10 to 13 carbon atoms and a fraction of normal paraffin fraction comprising 14 to 18 carbon atoms, a normal paraffin fraction comprising 5 to 9 carbon atoms, and a hydrogenated normal paraffin fraction comprising 19 to 35 carbon atoms.
The Fischer-Tropsch product stream as provided in step (a) is derived from a Fischer-Tropsch process. Fischer-Tropsch product stream is known in the art. By the term “Fischer-Tropsch product” is meant a synthesis product of a Fischer-Tropsch process. In a Fischer-Tropsch process the synthesis gas is converted to a synthesis product. The synthesis gas or syngas is obtained by conversion of a hydrocarbonaceous feedstock. Suitable feedstocks include natural gas, crude oil, heavy oil fractions, coal, biomass and lignite. A Fischer-Tropsch product derived from a hydrocarbonaceaous feedstock which is normally in the gas phase may also be referred to a GTL (Gas-to-Liquids) product. The preparation of a Fischer-Tropsch product has been described in e.g. WO2003/070857.
   Known to those skilled in the art is that the temperature and pressure at which the Fischer-Tropsch process is conducted influences the degree of conversion of synthesis gas into hydrocarbons and impacts the level of branching of the paraffins (thus amount of isoparaffins). Typically, the process for preparing a Fischer-Tropsch derived wax may be carried out at a pressure above 25 bara. Preferably, the Fischer-Tropsch process is carried out at a pressure above 35 bara, more preferably above 45 bara, and most preferably above 55 bara. A practical upper limit for the Fischer-Tropsch process is 200 bara, preferably the process is carried out at a pressure below 120 bara, more preferably below 100 bara.
      The Fischer-Tropsch process is suitably a low temperature process carried out at a temperature between 170 and 290° C., preferably at a temperature between 180 and 270° C., more preferably between 200 and 250° C.
   The conversion of carbon monoxide and hydrogen into hydrocarbons in the process according to the present invention may be carried out at any reaction pressure and gas hourly space velocity known to be suitable for Fischer-Tropsch hydrocarbon synthesis. Preferably, the reaction pressure is in the range of from 10 to 100 bar (absolute), more preferably of from 20 to 80 bar (absolute). The gas hourly space velocity is preferably in the range of from 500 to 25,000 h-1, more preferably of from 900 to 15,000 h-1, even more preferably of from 1,300 to 8,000 h-1. Preferably, the reaction pressure and the gas hourly space velocity are kept constant.
      The amount of isoparaffins is suitably less than 20 wt % based on the total amount of paraffins having from 9 to 24 carbon atoms, preferably less than 10 wt %, more preferably less than 7 wt %, and most preferably less than 4 wt %.
      Suitably, the Fischer-Tropsch derived product stream according to the present invention comprises more than 75 wt % of n-paraffins, preferably more than 80 wt % of n-paraffins. Further, the paraffin wax may comprise iso-paraffins and cyclo-alkanes.
      The Fischer-Tropsch process for preparing the Fischer-Tropsch derived product stream according the present invention may be a slurry Fischer-Tropsch process, an ebullated bed process or a fixed bed Fischer-Tropsch process, especially a multitubular fixed bed. Preferably, the Fischer-Tropsch process is a fixed bed Fischer-Tropsch process.
      The product stream of the Fischer-Tropsch process is usually separated into a water stream, a gaseous stream comprising unconverted synthesis gas, carbon dioxide, inert gasses and C1 to C4, and a C5+ stream. The Fischer-Tropsch product stream comprises preferably a wax and a liquid stream.
      The full Fischer-Tropsch hydrocarbonaceous product suitably comprises a C1 to C300 fraction.
      Lighter fractions of the Fischer-Tropsch product, which suitably comprises C1 to C4 fraction are separated from the Fischer-Tropsch product by distillation thereby obtaining a Fischer-Tropsch product stream, which suitably comprises C5 to C300 fraction.
      The above weight ratio of compounds having at least 60 or more carbon atoms and compounds having at least 30 carbon atoms in the Fischer-Tropsch product is preferably at least 0.2, more preferably 0.3.
      Suitably, in case of preparation of Fischer-Tropsch derived wax fraction having a congealing point of above 90° C. the above weight ratio is at least 0.5.
   The weight ratio in the Fischer-Tropsch product may lead to Fischer-Tropsch derived paraffin waxes having a low oil content.
   In step (b) of the process according the present invention the Fischer-Tropsch product of step (a) is separated to obtain a first gaseous hydrocarbon stream and a first liquid hydrocarbon stream. The separation in step (b) is suitably carried out at a temperature in a range of from 160 to 350° C., preferably from 190 to 250° C. and at a pressure in a range of from 5 to 150 bar. Also, the separation preferably takes place in the Fischer-Tropsch reactor.
   The first gaseous hydrocarbon stream as obtained in step (b) preferably comprises 1 to about 35 carbon atoms. Suitably, the first liquid hydrocarbon stream as obtained in step (b) comprises about 16 to 90 carbon atoms. The first liquid hydrocarbon stream can be considered as heavy product that is solid and ambient pressure and temperature. It can be seen that the carbon distribution of the first liquid hydrocarbon stream and the gaseous hydrocarbon stream as obtained in step (b) overlap partially as the partitioning in the two phases is related to the vapour pressure at conditions of separation. The starting point and the end point of the carbon distribution of liquid hydrocarbon stream have were determined using the 0.5 wt % cut off point of the hydrocarbon pool. Both the end point of the carbon distribution of the first gaseous stream and the starting point of the first liquid hydrocarbon stream can differ from the mentioned 35 and 16 depending on the conditions of separation.
      Hence, the end point of the carbon distribution of the first gaseous stream is in a range of from 25 to 50 carbon atoms, preferably from 35 to 40 and the starting point of the first liquid hydrocarbon stream is in a range of from 5 to 25 carbon atoms, preferably from 10 to 20 carbon atoms.
   In step (c) of the process according to the present invention the first gaseous hydrocarbon stream of step (b) is cooled and separated in one or more steps to obtain a second liquid hydrocarbon steam and a third liquid hydrocarbon stream.
   Cooling and separation in step (c) of the first gaseous hydrocarbon stream of step (b) is suitably carried out at a temperature in a range of from 5 to 180° C. and at a pressure in a range of from 5 to 145 bar.
   Preferably, the first gaseous hydrocarbon stream of step (b) is cooled and separated in two steps in step (c).
   Suitably, first the first gaseous hydrocarbon stream of step (b) is cooled to obtain a second gaseous hydrocarbon stream and a second liquid hydrocarbon stream followed by the cooling of the second gaseous hydrocarbon stream to obtain a third gaseous hydrocarbon stream and a third liquid hydrocarbon stream.
   Cooling of the second gaseous hydrocarbon stream is also carried out at the conditions as described above for the cooling in step (c).
      Preferably, the second liquid hydrocarbon stream comprises 5 to 30 carbon atoms. The second gaseous hydrocarbon stream suitably comprises 1 to 25 carbon atoms. Also, the third gaseous hydrocarbon stream comprises 1 to 4 carbon atoms. The third liquid hydrocarbon stream suitable comprises 3 to 20 carbon atoms.
   In step (d) of the process according to the present invention the second and third liquid hydrocarbon stream of step (c) is subjected to a hydrogenation step, thereby obtaining a hydrogenated liquid hydrocarbon stream.
      The hydrogenation is suitably carried out at a temperature between 200 and 275° C. and at a pressure between 20 and 70 bar. Typically, hydrogenation removes olefins and oxygenates from the fractions being hydrogenated. The amount of oxygenates in the streams prior to hydrogenation is less than 5 ppm (mg/kg). Oxygenates are preferably hydrocarbons containing one or more oxygen atoms per molecule.
      Typically, oxygenates are alcohols, aldehydes, ketones, esters, and carboxylic acids.
      Typically, prior to the hydrogenation step in (d) the second and the third liquid hydrocarbon liquid stream are combined. Also, prior to the hydrogenation step in (d) a fraction comprising 5 to 9 carbon atoms is separated from the second and the third liquid hydrocarbon steam by atmospheric distillation.
      In step (e) of the process according to the present invention the hydrogenated liquid hydrocarbon stream of step (d) is separated by one or more atmospheric distillation(s), thereby obtaining a hydrogenated normal paraffin fraction comprising 5 to 9 carbon atoms, a hydrogenated normal paraffin fraction comprising 10 to 13 carbon atoms, a hydrogenated normal paraffin fraction comprising 14 to 18 carbon atoms, and a hydrogenated normal paraffin fraction comprising 19 to 35 carbon atoms.
Preferably, the atmospheric distillation in step (e) is at a temperature in the range of 200 to 400, preferably 300 to 350° C.

The normal paraffin comprising from 10 to 13 carbon atoms is also known as light detergent fraction (LDF) and the normal paraffin comprising from 14 to 18 carbon atoms is also known as heavy detergent fraction (HDF).

Preferably, the normal paraffin fraction comprising 10 to 13 carbon atoms has a flashpoint according to ASTM D93 between 70 and 80° C., more preferably between 70 and 75° C. and most preferably a flashpoint of 74° C. Also the kinematic viscosity according to ASTM D445 of the normal paraffin fraction comprising 10 to 13 carbon atoms at 40° C. is between 1.30 and 1.45 cSt, preferably between 1.30 and 1.40 cSt, most preferably the kinematic viscosity at 40° C. of LDF is 1.36 cSt.

The pour point of the normal paraffin fraction comprising 10 to 13 carbon atoms according to ASTM D97 is below 0° C., preferably below −15° C., more preferably below −20° C. and most preferably below −21° C.

The density of the normal paraffin fraction comprising 10 to 13 carbon atoms according to ASTM D1298 is between 700 and 800 kg/m³, preferably between 700 and 750 kg/m³.

Preferably, the normal paraffin fraction comprising 14 to 18 carbon atoms has a flashpoint between 100 and 130° C., more preferably between 110 and 125° C. and most preferably a flashpoint of 122° C. Also the kinematic viscosity of the normal paraffin fraction comprising 14 to 18 carbon atoms according to ASTM D445 at 40° C. is between 2.00 and 3.00 cSt, preferably between 2.50 and 2.70 cSt, most preferably the kinematic viscosity at 40° C. of the normal paraffin fraction comprising 14 to 18 carbon atoms is 2.66 cSt.

The pour point of the normal paraffin fraction comprising 14 to 18 carbon atoms according to ASTM D97 is below 20° C., preferably below 15° C., more preferably below 12° C.

The density of the normal paraffin fraction comprising 14 to 18 carbon atoms according to ASTM D1298 is between 700 and 800 kg/m³, preferably between 750 and 780 kg/m³.

Suitably, the normal paraffin fraction comprising 10 to 13 carbon atoms has a flashpoint between 70 and 80° C., a kinematic viscosity according to ASTM D445 at 40° C. between 1.30 and 1.45 cSt, a pour point according to ASTM D97 below −21° C., and a density according to ASTM D1298 between 700 and 800 kg/m³.

In addition, the normal paraffin fraction comprising 14 to 18 carbon atoms has a flashpoint between 100 and 130° C., a kinematic viscosity according to ASTM D445 at 40° C. between 2.00 and 3.00 cSt, a pour point according to ASTM D97 is below 12° C., and a density according to ASTM D1298 between 700 and 800 kg/m3.

In another aspect, the present invention provides a process to prepare linear alkyl-benzene sulphonate using a normal paraffin fraction comprising 10 to 13 carbon atoms as obtained according to the process of the present invention.

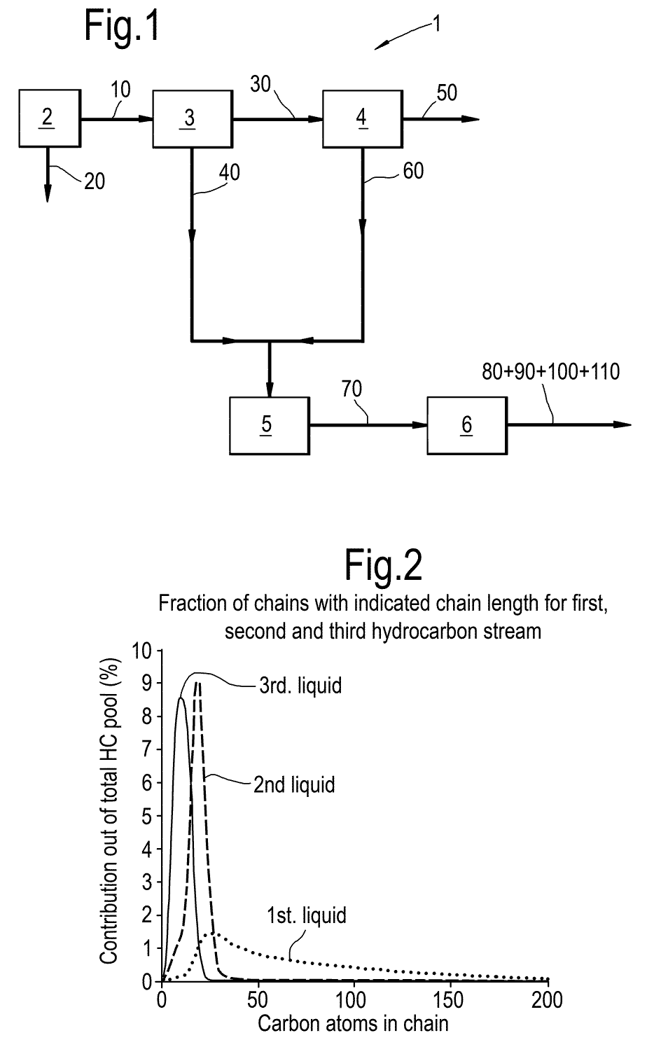

FIG. 1 schematically shows a process scheme of the process scheme of a preferred embodiment of the process according to the present invention.

For the purpose of this description, a single reference number will be assigned to a line as well as a stream carried in that line.

The process scheme is generally referred to with reference numeral 1.

In a Fischer-Tropsch process reactor 2 a Fischer-Tropsch product stream is obtained. Separation into a first gaseous hydrocarbon stream 10 and a first liquid fraction 20 is accomplished in the reactor itself.

The gaseous hydrocarbon stream 10 is fed to a cooling unit 3 wherein the gaseous hydrocarbon stream is cooled and separated to obtain a second gaseous hydrocarbon stream 30 and a second liquid fraction 40. The gaseous hydrocarbon stream 30 is fed to another cooling unit 4 wherein the gaseous hydrocarbon stream 30 is cooled and separated to obtain a third gaseous hydrocarbon stream 50 and a third liquid fraction 60. The second liquid fraction 40 and the third liquid fraction 60 are fed to a hydrogenation reactor 5 to obtain a hydrogenated liquid hydrocarbon stream 70. The hydrogenated liquid hydrocarbon stream 70 is distilled in one or more atmospheric distillation columns 6 to recover a hydrogenated normal paraffin fraction 80 comprising 5 to 9 carbon atoms, a fraction 90 comprising 10 to 13 carbon atoms, a fraction 100 comprising 14 to 18 carbon atoms and a fraction 110 comprising 19 to 35 carbon atoms.

The invention is illustrated by the following non-limiting examples.

The invention is illustrated by the following non-limiting examples.

### EXAMPLE 1

Product Distribution of the First, Second and Third Liquid Hydrocarbon Streams

In Table 1 the flows of molecules with indicated chain length in three liquid hydrocarbon streams is given, with full distribution of the streams depicted in FIG. 2. The first liquid stream is obtained at a pressure of 55 bar and a temperature of 215° C., the second liquid stream at a pressure of 55 bar and a temperature of 159° and the third liquid stream at a pressure of 53 bar and a temperature of 15° C. It can be seen that the majority of the normal paraffins is present in the combined stream of 2^{nd} and 3^{rd} liquid.

TABLE 1
Paraffin content in 1^{st}, 2^{nd} and 3^{rd} liquid hydrocarbon streams.

| | | | | Fraction |
|---|---|---|---|---|
| | | | | in 2^{nd} |
| | 1^{st} | 2^{nd} | 3^{rd} | and 3^{rd} |
| | liquid | liquid | liquid | liquid |
| C10 | 2 | 2 | 35 | 94% |
| C11 | 3 | 2 | 34 | 93% |
| C12 | 3 | 3 | 33 | 92% |
| C13 | 4 | 4 | 31 | 89% |
| C14 | 6 | 5 | 27 | 85% |
| C15 | 8 | 7 | 23 | 80% |
| C16 | 10 | 9 | 18 | 73% |
| C17 | 13 | 11 | 13 | 65% |
| C18 | 15 | 12 | 9 | 57% |

### EXAMPLES 2 TO 3

Process to Prepare Normal Paraffins

In the comparative example 2 all the liquid hydrocarbon streams are combined and after hydrogenation used for the production of normal paraffins. The case according to the invention is represented by example 3. In Table 2 the total size of the hydrocarbon streams is indicated as well as the split in C10−, target range of normal paraffins and C18+. Comparison of example 3 (according to the invention) with comparative example 2 it can be seen that the paraffin total yields were only 16% lower. However, for the comparative example the amount of feed could be reduced with as much as 76%. This enables to build the Hydrogenation Reactor and it's surrounding equipment 4 times smaller. This brings a very significant reduction in cost to build the equipment and is combined with lower energy consumption for operation. On top of that, the very low amount of heavier hydrocarbons in example 3 enables to do the final distillation at atmospheric conditions, whereas an expensive vacuum distillation operation in combination with an atmospheric distillation (to remove the lighter components) is required for the comparative example. Hence the situation as per invention in example 2 is much more attractive as the expensive vacuum distillation that requires high energy loads, could be eliminated.

TABLE 2
Liquid hydrocarbon streams in tpd for indicated fractions.

| Example | Liquids | C10− | C10-C17 | C18+ | Total |
|---|---|---|---|---|---|
| 2 | 1 + 2 + 3 | 193 | 307 | 1781 | 2280 |
| 3 | 2 + 3 | 180 | 258 | 97 | 535 |

### EXAMPLE 4

Process to Prepare C10 to 13 and C14 to 18 Normal Paraffins

The recovered paraffins will need further distillation to meet the product specification of the lighter C10-C13 normal paraffins and C14-C18 normal paraffins final products. The resulting compositions are given in Table 3 and Table 4. It can be seen that the compositions according to the invention are lighter compared to the comparative example.

TABLE 3
Composition of LDF for comparative example
2 and example 3 as per invention

| Ex. | C9 | C10 | C11 | C12 | C13 | C14 | Mw |
|---|---|---|---|---|---|---|---|
| 2 | 0.2 | 10 | 32.0 | 31.9 | 25.5 | 0.5 | 166.4 |
| 3 | 0.2 | 10 | 32.2 | 31.6 | 25.5 | 0.5 | 166.3 |

TABLE 4
Composition of HDF for comparative example
2 and example 3 as per invention

| Ex. | C13 | C14 | C15 | C16 | C17 | C18 | Mw |
|---|---|---|---|---|---|---|---|
| 2 | 0.5 | 23.8 | 24.3 | 23.4 | 22.6 | 5.5 | 220.4 |
| 3 | 0.5 | 26.7 | 25.7 | 22.4 | 19.3 | 5.5 | 218.9 |

In Table 5 the normal paraffin final product weight fractions are given. It can be seen that in example 3 according to the invention almost the same amount of LDF is obtained, with a lower amount of HDF. The LDF content out of the NP products increases hence from 44 to 49%.

It is advantaged to have a larger amount of LDF, because this stream can be used very well for the production of LAB. The NP consumption for LAB production is significant and the NP has a good premium compared to kerosene.

TABLE 5
Volumetric split of NP in LDF and HDF for
comparative example 2 and example 3 as per invention

| | LDF | HDF | | |
|---|---|---|---|---|
| | (tpd) | (tpd) | LDF | HDF |
| 2 | 122 | 155 | 44% | 56% |
| 3 | 113 | 118 | 49% | 51% |

1. A normal paraffin composition comprising from 45 to 60 wt. % of a fraction of normal paraffin having from 10 to 13 carbon atoms and from 40 to 55 wt. % of a fraction of normal paraffin having from 14 to 18 carbon atoms.

2. The normal paraffin composition according to claim 1, comprising 49 wt. % of the fraction of normal paraffin having from 10 to 13 carbon atoms and 51 wt. % of a fraction of normal paraffin having from 14 to 18 carbon atoms based on the total amount of the normal paraffin composition.

3. The normal paraffin composition according to claim 1, wherein the fraction of normal paraffin having from 10 to 13 carbon atoms comprises 10 carbon atoms in the range of from 10 to 11 wt. %, 11 carbon atoms in the range of from 30 to 32 wt. %, 12 carbon atoms in a range of from 30 to 32 wt. % and 13 carbon atoms in a range of from 23 to 26 wt. % based on the amount of the normal paraffin having from 10 to 13 carbon atoms.

4. The normal paraffin composition according to claim 1, wherein the fraction of normal paraffin having from 14 to 18 carbon atoms comprises 14 carbon atoms in a range of from 25 to 27 wt. %, 15 carbon atoms in a range of from 24 to 26 wt. %, 16 carbon atoms in a range of from 22 to 23 wt. %, 17 carbon atoms in a range of from 18 to 20 wt. % and 18 carbon atoms in a range of from 4 to 6 wt. % based on the amount of the normal paraffin having from 14 to 18 carbon atoms.

5. The normal paraffin composition according to claim 1, wherein the normal paraffin is a Fischer-Tropsch derived normal paraffin composition.

6. A process to prepare a normal paraffin composition comprising the following steps:
(a) providing a Fischer-Tropsch product stream;
(b) separating the Fischer-Tropsch product stream of step (a), thereby obtaining a gaseous hydrocarbon stream and a first liquid hydrocarbon stream;
(c) cooling of the gaseous hydrocarbon stream of step (b) in one or more steps to obtain a second liquid hydrocarbon stream and a third liquid hydrocarbon stream;
(d) subjecting the second and third liquid hydrocarbon streams of step (c) to a hydrogenation step, thereby obtaining a hydrogenated liquid hydrocarbon stream;
(e) separating the hydrogenated liquid hydrocarbon stream of step (d) by one or more atmospheric distillation(s), thereby obtaining at least a normal paraffin composition comprising a fraction of normal paraffin fraction comprising 10 to 13 carbon atoms and a fraction of normal paraffin fraction comprising 14 to 18 carbon atoms, a normal paraffin fraction comprising 5 to 9 carbon atoms, and a hydrogenated normal paraffin fraction comprising 19 to 35 carbon atoms.

7. The process according to claim 6, wherein the normal paraffin fraction comprising 10 to 13 carbon atoms has a flashpoint according to ASTM D93 between 70 and 80° C., a kinematic viscosity according to ASTM D445 at 40° C. between 1.30 and 1.45 cSt, a pour point according to ASTM D97 below −21° C., and a density according to ASTM D1298 between 700 and 800 kg/m3.

8. The process according to claim 6, wherein the normal paraffin fraction comprising 14 to 18 carbon atoms has a flashpoint according to ASTM D93 between 100 and 130° C., a kinematic viscosity according to ASTM D445 at 40° C. between 2.00 and 3.00 cSt, a pour point according to ASTM D97 is below 12° C., and a density according to ASTM D1298 between 700 and 800 kg/m3.

9. A process to prepare linear alkyl-benzene sulphonate using a normal paraffin fraction comprising 10 to 13 carbon atoms.
